(19) United States
(12) Patent Application Publication
(10) Pub. No.: US 2023/0049673 A1
(43) Pub. Date: Feb. 16, 2023

(54) ENDOSCOPE WITH REUSABLE OPTICAL AND ELECTRICAL DISTAL ASSEMBLY

(71) Applicant: RHB Holdings LLC,, Lincoln, MA (US)

(72) Inventors: Yunfei Cao, Lincoln, MA (US); Dwight Meglan, Westwood, MA (US); Ann Xin, Newark, CA (US)

(21) Appl. No.: 17/403,818

(22) Filed: Aug. 16, 2021

(51) Int. Cl. A61B 1/05; A61B 1/06; A61B 1/00; A61B 1/005

(52) U.S. Cl. CPC A61B 1/05; A61B 1/0676; A61B 1/00124; A61B 1/00098; A61B 1/0057; A61B 1/0684; A61B 1/00066

(57) ABSTRACT

An endoscope that includes an elongated bougie, a distal tip at a distal end of the bougie and a handle at a proximal end of the bougie. The distal tip has a camera, an LED light, and a distal tip housing forming a distal tip chamber. The endoscope also has an electronic connector, such as a PCB board connector, having at least one PCB board, and at least a portion of the PCB board is nestled within the distal tip chamber. The PCB board connector is disposed between the distal tip and the distal end of the elongated bougie when being assembled. The distal tip and the PCB board connector are attached into one integral distal assembly by removable attaching means, such as a pair of snap fit hood and holder. The integral distal end assembly is configured to be cut from the old bougie used in a medical procedure and to be collected and later disassembled, sterilized, and reassembled with another new bougie.

### TECHNICAL FIELD

This application relates generally to endoscopes, particularly to endoscopes with bougie distal tips hosting optical and electrical parts to be reused and further to methods of collecting and reusing bougie distal tips of endoscopes.

### BACKGROUND

Generally, the cost of medical procedures for minimally, invasively exploring and treating the body of a subject is significantly impacted by the cost of the endoscopes or scope used. Such scopes can be used, for example, for imaging and treating issues involving the kidney calyces, bladder, ureter, or airway intubation. In addition to the significant cost of such reusable scopes, they must be cleaned and sterilized after each procedure to prevent cross infection of patients. This process has been shown to be very costly as well. For a high throughput endoscopy center, this means having to stock several scopes to do multiple procedures in a day as well as having to stock additional scopes to supplement the instrument due to breakage.

The most expensive parts of an endoscope include the camera, light source and the PCB board. Some disposable endoscopes require complicated assembly, sterilization and reassembly processes in order to reuse one or more costly items. For these reasons, an endoscope assembly that allows for easy and effective sterilization and reuse of the most expensive parts may reduce the cost of endoscopy use per procedure and meet clinician quality expectations. It also leads to improvements in both patient safety and hospital efficiency. Accordingly, herein disclosed methods and apparatus are directed to solve one or more problems set forth above and other problems.

### SUMMARY

In accordance with a first aspect of the present disclosure, there is set forth an endoscope that includes an elongated bougie, a distal tip at a distal end of the bougie and a handle at a proximal end of the bougie. The distal tip has a camera, an LED light, and a distal tip housing forming a distal tip chamber. The endoscope also has a PCB board connector having at least one PCB board, and at least a portion of the PCB board is nestled within the distal tip chamber. The PCB board connector is disposed between the distal tip and the distal end of the elongated bougie when being assembled. The distal tip and the PCB board connector are attached into one integral distal assembly by removable attaching means, such as a pair of snap fit hood and holder. The integral distal end assembly is configured to be cut from the old bougie used in a medical procedure and to be collected and later disassembled, sterilized, and reassembled with another new bougie.

Also disclosed is a distal assembly of an endoscope that includes a distal tip having a camera, an LED light, and a distal tip housing forming a distal tip chamber. The distal assembly also includes a PCB board connector having at least one PCB board. At least a portion of the PCB board is configured to nestle within the distal tip chamber. The distal tip and the PCB board connector are attached into one integral distal assembly by removable attaching means, such as a pair of snap fit hood and holder.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosed systems and methods and are not intended as limiting. For purposes of clarity, not every component may be labeled in every drawing. In the following description, various embodiments are described with reference to the following drawings.

FIG. 1 is a front view of an endoscope with a reusable optical and electrical distal end in accordance with the present disclosure.

FIG. 2 is an enlarged front view of the optical and electrical distal end of the endoscope in accordance with the present disclosure.

FIG. 3 is an exploded view of the optical and electrical distal end of the endoscope in accordance with the present disclosure.

FIG. 4. is a more detailed front view of the endoscope with the reusable optical and electrical distal end in accordance with the present disclosure.

FIG. 5 is a longitudinal cross-sectional view of the endoscope in accordance with the present disclosure.

FIG. 6 is a side view of the endoscope with the reusable optical and electrical distal end in accordance with the present disclosure.

FIG. 7 is longitudinal cross-sectional view of the endoscope with the reusable optical and electrical distal end in accordance with the present disclosure.

FIG. 8 is another side view of the endoscope with the reusable optical and electrical distal end in accordance with the present disclosure.

FIG. 9 is longitudinal cross-sectional view of the endoscope with the reusable optical and electrical distal end in accordance with the present disclosure.

FIG. 10 is another front view of the reusable optical and electrical distal end in accordance with the present disclosure.

FIG. 11 is another longitudinal cross-sectional view of the endoscope with the reusable optical and electrical distal end in accordance with the present disclosure.

FIGS. 12-14 are circumferential cross-sectional views of the reusable optical and electrical distal end at difference respective cut-lines in accordance with the present disclosure.

### DETAILED DESCRIPTION

### List of Nomenclatures

Numerals corresponding to those shown in the drawings are listed in Table-1. Terms assigned to corresponding numerals are also given in Table-1.

TABLE 1
Numerals and corresponding terms used in the present disclosure.

| | |
|---|---|
| 1 | endoscope |
| 2 | distal assembly |
| 20 | distal tip |
| 22 | camera |
| 23 | camera slot |
| 24 | light source (LED) |
| 26 | PCB connector |
| 28 | bougie (bougie) |
| 30 | bougie proximal end |
| 32 | handle |
| 34 | articulation knob |
| 35 | pull wire |
| 36 | vertebral column |
| 38 | PCB board |
| 39 | electric pads - power supply and data |
| 40 | PCB connector housing |
| 42 | distal end housing |
| 43 | connector chamber front |
| 44 | connector chamber back |
| 46 | PCB connector |
| 48 | PCB line |
| 50 | snap fit hook |
| 52 | snap fit holder |
| 54 | handle housing |
| 56 | external electronic connector Type C |
| 58 | pulley |
| 59 | pull wire knot |
| 60 | vertebra sheathing |
| 62a | first vertebrae slit |
| 62b | second vertebrae slit |
| 62 | vertebrae slit |
| 64 | bougie conduit |
| 66 | pull wire channel |
| 68 | opening tool notch |
| 70 | pointing guide |

The following description of the vertebral column of the endoscopes uses an ureteroscope as an example. It should be appreciated that the scope and spirit of this disclosure is not limited to this example. The example of using partially reusable endoscopes, entirely reusable, or entirely disposable endoscopes do not affect the scope of present disclosure. The term of endoscope or scope can be interchangeable used with many types of endoscopes, such as ureteroscope, cystoscope, bronchoscope, and laparoscope, etc.

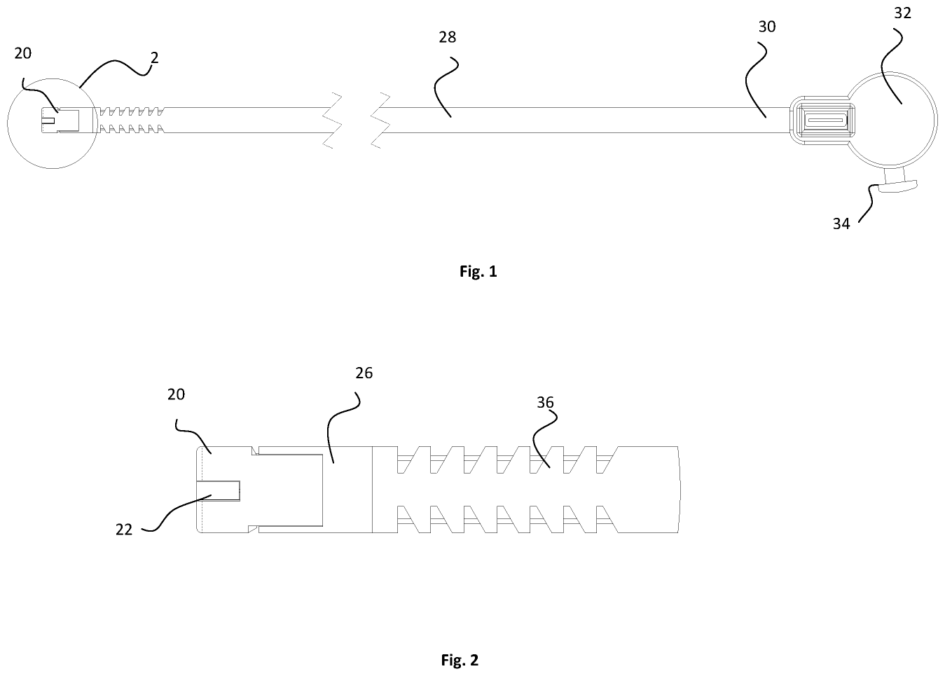

FIG. 1 is a front view of an endoscope with a reusable optical and electrical distal assembly in accordance with the present disclosure. Endoscope 1 includes a probe handle 32 and a probe 28 which is exchangeably called a bougie or an elongated bougie 28 as it often takes a thin and elongated shape to assist the insertion into human or animal bodies to provide investigation and/or treatment for the targeted organs. Endoscope 1 can also be called a video bougie which may include a video display (not shown) displaying data captured by bougie 28.

FIG. 2 is an enlarged front view of the optical and electrical distal end of the endoscope in accordance with the present disclosure.

Referring to FIGS. 1 and 2, bougie 28 is an elongated conduit or tube, often being flexible. Probe 28 includes distal assembly 2, a vertebral column 36 and bougie 28. Distal assembly 2 includes a distal tip 20 and a PCB connector 26 (both described later in greater detail). Bougie 28 is rigid enough to be pushed through to guide the bougie to enter into body cavities or organs. Vertebral column 36 is also rigid enough to be pushed through to guide the bougie to enter into body cavities or organs yet passively flexible to change direction to where the inspection is needed to take place. Bougie 28 includes a proximal end 30 connecting to handle 32.

It should be noted that PCB connector 26 can be any kind of electronic connectors, such as a pair of USB-C connector and socket or any of other choices. The variations of such are within the scope of the present disclosure.

Endoscope 1 further includes an articulation knob 34 disposed at handle 32. Upon being maneuvered, articulation knob 34 causes a deflection of vertebral column 38, which is connected with the articulation knob 34 by a pulling assembly explained later in more detail.

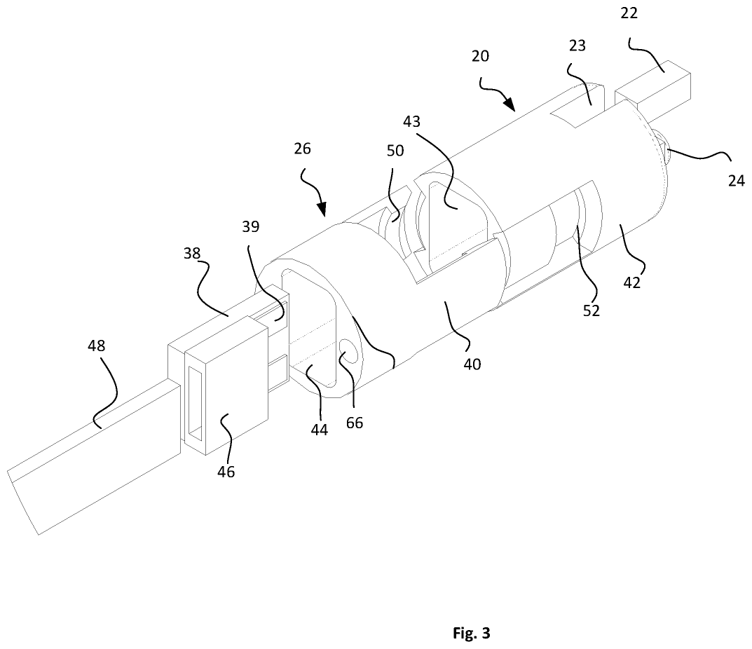

Referring to FIGS. 2 and 3, endoscope 1 includes PCB connector 26 at the distal assembly 2, connecting distal tip 20 and the body of bougie 28. Distal tip 20 includes a camera 22 and a light source, such as an LED 24. As shown in this example embodiment, PCB connector 26 abuts distal tip 20 and connects distal tip 20 with vertebral column 36.

In one example embodiment, vertebral column 36 and the bougie 28 are made by the same process of plastic formation, such as plastic molding or plastic extrusion.

As such, this configuration enables one of the novel aspects of the present disclosure which allows for the most expensive electronic and optical parts of endoscope 1 to be included as an integral part and all located at distal assembly 2, which can be recollected after a surgery, sterilized and reused. On the other hand, the rest of endoscope 1, including bougie 28, probe proximal end 30, and handle 32, which are mostly plastic parts, can all be disposed after each use. This is different from other existing reusable or partially reusable endoscope, in which, the PCB assembly is located at or near the handle portion of an endoscope, preventing it from being easily recollected, sterilized and reused together with the camera and LED portions.

FIG. 3 is an exploded view of the optical and electrical distal assembly of the endoscope in accordance with the present disclosure.

Referring to FIG. 3, distal assembly 2 includes two major portions: distal tip 20 and PCB connector 26. Distal tip 20 includes a distal assembly housing 42 having a camera slot 23 at its far distal end, shaped to receive and host camera 22. Distal tip 20 also includes a light source, for example, an LED 24 disposed at the distal end of distal end housing 42.

Distal end housing 42 takes a hollow structure, forming a connector chamber front 43 to host PCB board 38. Connector chamber front 43 may take any shape, such as a rectangular prism or a cylindrical prism, according to the shape of PCB board 38.

Reference is continuously made to FIG. 3. Likewise, PCB connector 26 includes a PCB connector housing 40, taking a hollow structure, forming a connector chamber back 44 to host PCB connector 46 and or part of PCB board 38. Connector chamber back 43 also may take any shape, such as a rectangular prism or a cylindrical prism, according to the shape of PCB board 38.

It can be noted in FIG. 3 that distal end housing 42 and PCB connector housing 40 have compatible shapes that, when assembled, they take an integral shape, together forming distal assembly 2. Distal end housing 42 and PCB connector housing 40 further comprise a detachable attachment assembly, such as a pair of snap fit hook 50 and snap fit holder 52. In this example embodiment, snap fit hook 50 and snap fit holder 52 both take the compatible matching shape of a circular rim. When assembled, snap fit hook 50 and snap fit holder 52 hold distal tip 20 and PCB connector 26 firmly as an integral piece during surgical procedures. There is also sealing meaning, such as Teflon or o-ring (not shown) applied along abutting location between distal tip 20 and PCB connector 26 to prevent any leaking and contamination from body fluid.

As can be seen, PCB board connector 26 and the distal tip 20 have a pair of matching removably attaching means causing the distal tip 20 and the PCB board connector 26 to be removably attached as an integral distal end when assembled.

Referring to FIG. 11 and continuously referring to FIG. 3, PCB board 38 in this example embodiment collectively serves for any or all electronic and power connection need. For example, one or a couple of the connecting components, shown in FIG. 11, may serve as power supply connector. Other connecting components of PCB board 38 may serve as data connecting means. The PCB board connector housing 40 holds at least partially the PCB board 38, and the PCP board connector housing 40 is configured to be removably attached to the distal tip to electronically connect the PCB board and/or power with the sensor and the light source when assembled.

Referring to FIGS. 1 and 3, endoscope 1 further includes a PCB line 48, which is cabled through bougie 28 between handle 32 and PCB connector 26, connecting PCB connector 26 via a PCB connector 46.

As can be seen, PCB connector housing 40 is configured to be pulled away co-axially with the distal end housing, from the connector chamber front 43, bringing the PCB board away from the distal tip 20 when disassembled or to be pushed into connector chamber front 43 co-axially, causing the PCB board to be nestled within the distal tip chamber.

In the example embodiment, when assembled, PCB board 38, connector 46, camera 22, and light source 24 are electronically connected, and at least major portion of them are all housed in distal tip 20. Distal tip 20 and PCB connector 26 form one integral piece, which is the distal assembly 2 as shown in FIG. 1. At the completion of a surgical or examination medical procedure, this allows for the integral distal assembly 2, which contains the most expensive electronic and optical parts of endoscope 1 to be easily cut off from the rest of the bougie, collected at the site of the medical procedure and later batch sterilized for being reassembled into new endoscopes. The batch sterilization and reassembly does not need to be taken place on the hospital premises. The rest of endoscope 1, including bougie 28, probe proximal end 30, and handle 32, which are mostly plastic parts, can all be disposed after each use. When the endoscope according to the present disclosure is reassembled, a new and never used bougie 28, probe proximal end 30, and handle 32 are fit with the sterilized and reused distal assembly 2 into a new complete of endoscope.

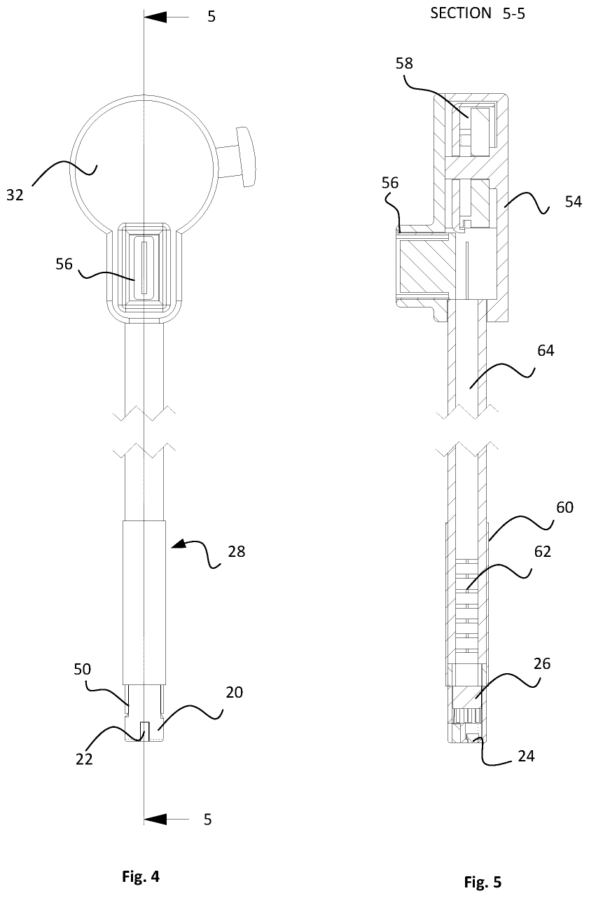

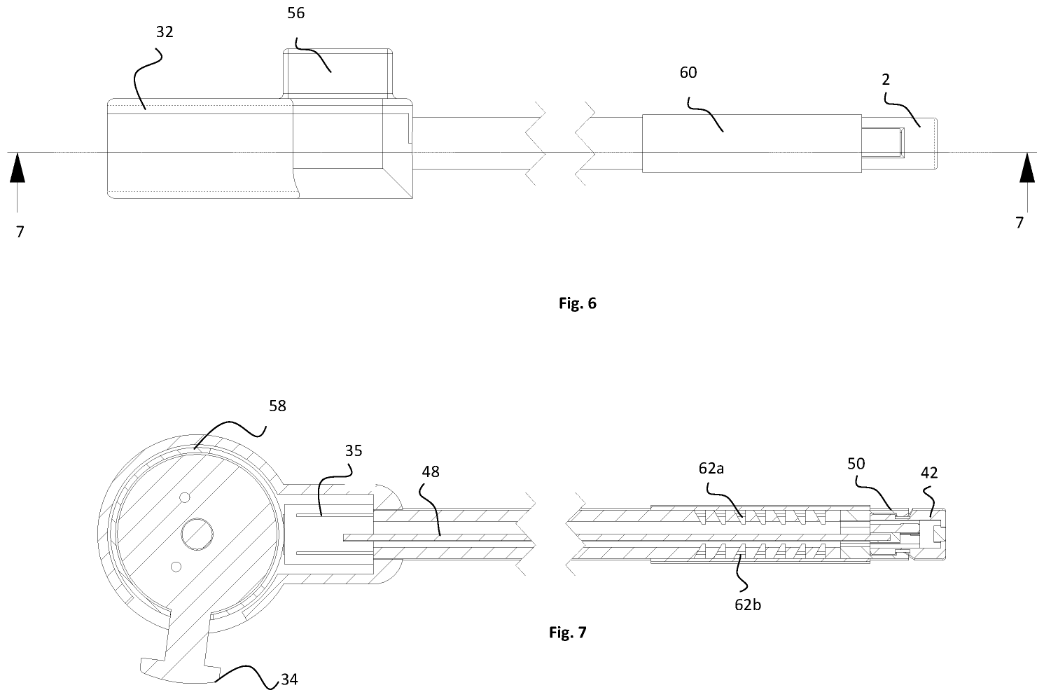

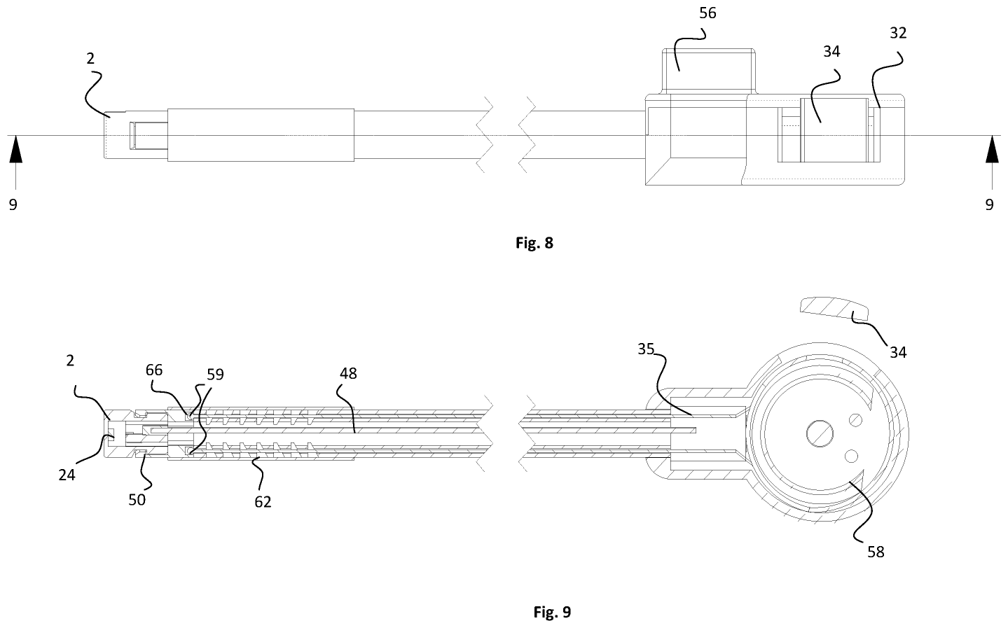

Reference now is made to FIGS. 4-9 which include more detailed front and side views and cross-sectional views of the endoscope with the reusable optical and electrical distal end in accordance with the present disclosure. FIG. 4 is a front view of the endoscope. FIG. 5 is a cross-section viewed at a cross section at line 5-5 in FIG. 4, with viewing direction indicated. FIG. 6 is side view of the endoscope. FIG. 7 is a cross-section viewed at a cross section at line 7-7 in FIG. 6, with viewing direction indicated in FIG. 6. FIG. 8 is another side view of the endoscope. FIG. 9 is a cross-section viewed at a cross section at line 9-9 in FIG. 8, with viewing direction indicated in FIG. 8.

As shown in FIGS. 4-9, handle 32 is formed by a handle housing 54, which encloses a wire pulley 58. Wire pulley 58 facilitates a mechanical assembly to cause the deflection of vertebral column 36 upon articulation knob 34 being maneuvered and pull wire 35 is pulled. Bougie 28, in many embodiments, are rigidly flexible and distal tip 16 is steerable.

It can be noted that vertebral column 36 has gaping slits on a circumferential surface of the vertebral column 36. Gaping slits opposite to the direction of the deflection open wider while the others close narrower during deflection.

As shown in FIG. 7, pull wire 35 may be looped around pulley 58 or belts around half circumference of pulley 58 on a side opposite to bougie proximal end 30, before entering into bougie proximal end 30, then continues into bougie 28 reaching vertebral column 36.

As shown in FIG. 9, on distal end of vertebral column 36, pull wire 35 may be attached to the end portion of vertebral column 36. In the example embodiment, pull wire 35 is tied to form a knot 59 to hold pull wire in place of a pull wire holder, which may be as simple as a holed short cylinder, allowing pull wire 35 to be threaded through, the size of which is configured to be smaller than knot 59. Alternatively, pull wire holder (not shown) may be a spring-loaded clamp that can be opened and closed to hold the end of pull wire 35. Further alternatively, pull wire 35 with one end having a crimp of a diameter larger than the hole of wire holder can be threaded into the wire holder from the other smaller end. All such alternations are within the scope of the present disclosure.

As such, when articulation knob 34 is swung, resulting pulley 58 to swing, with pull wire being flexible but not stretchable, it causes pull wire 35 to pull distal tip 20 away from the axial direction, further causing a deflection of vertebral column 36.

As shown in FIG. 3, on proximal end of PCB connector housing 40, it includes a pair of holes 66, configured to correspondingly accommodate pull wire knot 59 in this example embodiment.

As shown in FIG. 5, bougie 28 also includes vertebrae sheathing 60, enclosing and sealing vertebral column 36 from body fluid during surgical procedures.

Distal tip 20 also includes a slanted pointing guide at the furthest distal end, serving to ease and guide the inserting of bougie 28.

PCB line 48 shown in FIG. 3 is also shown to be cabled longitudinally through bougie 28 in FIG. 7. It connects PCB connector 46 electronically with an external electronic connector 56 deposited near handle 32. The housing or external electronic connector 56 can be an integral part of handle housing 54 shown in FIG. 5.

External electronic connector 56 can host and be connected with any external devices to transmit signals and/or power between PCB line 48 and the external electronics, such as a user interface, a video display device, or a power source.

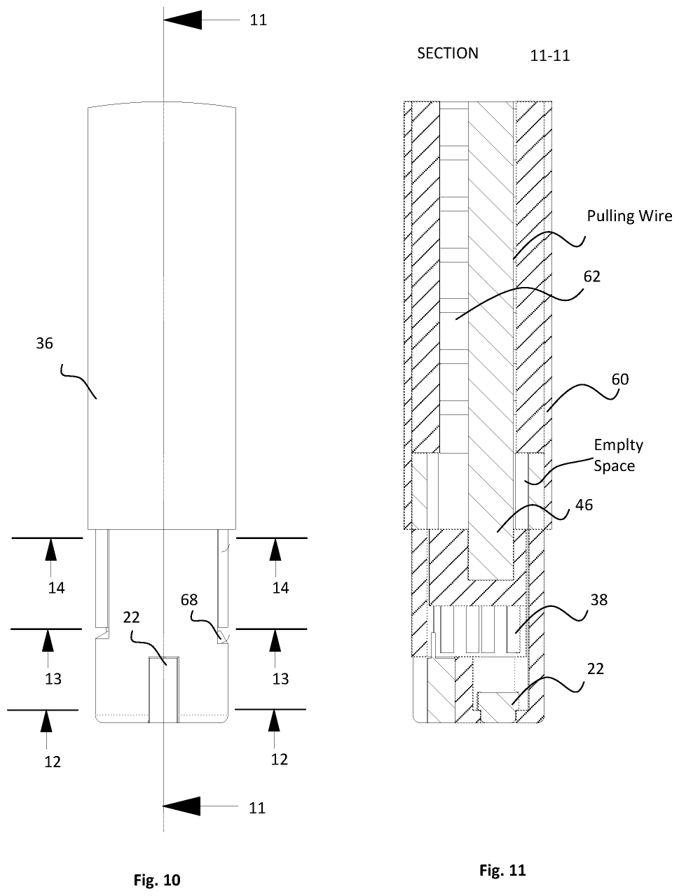

FIG. 10 is an elaborated front view of the reusable optical and electrical distal end in accordance with the present disclosure. FIG. 11 is a cross-section viewed at a cross section at line 11-11 in FIG. 10, with viewing direction indicated.

As shown in FIG. 10, distal assembly 2 further includes an opening tool notch 68 configured between distal tip 20 and PCB connector 26. Opening tool notch 68 is configured in the example embodiment to provide access to tools to pry and open the snap fit hook 50 (shown in FIGS. 3 and 9) during the process of sterilization and reassembly.

As shown in FIG. 11, PCB board 38 may include a small electronics circuit board, and/or connection contacts for power lines. The circuit board may include a processor that processes the image data capture by an image sensor or camera 22 at the tip of the endoscope. The processor coverts the image data from the format output to the standard image format such as HDMI that can be displayed on typical monitors.

In another example embodiment, camera 22 itself includes a processor that digitizes image signals and directly outputs digitized image data. All such alternative embodiments are within the scope of the present disclosure.

Yet in another example embodiment, distal tip 20 is an integral part including camera 24 and LED 26 without the need to assemble.

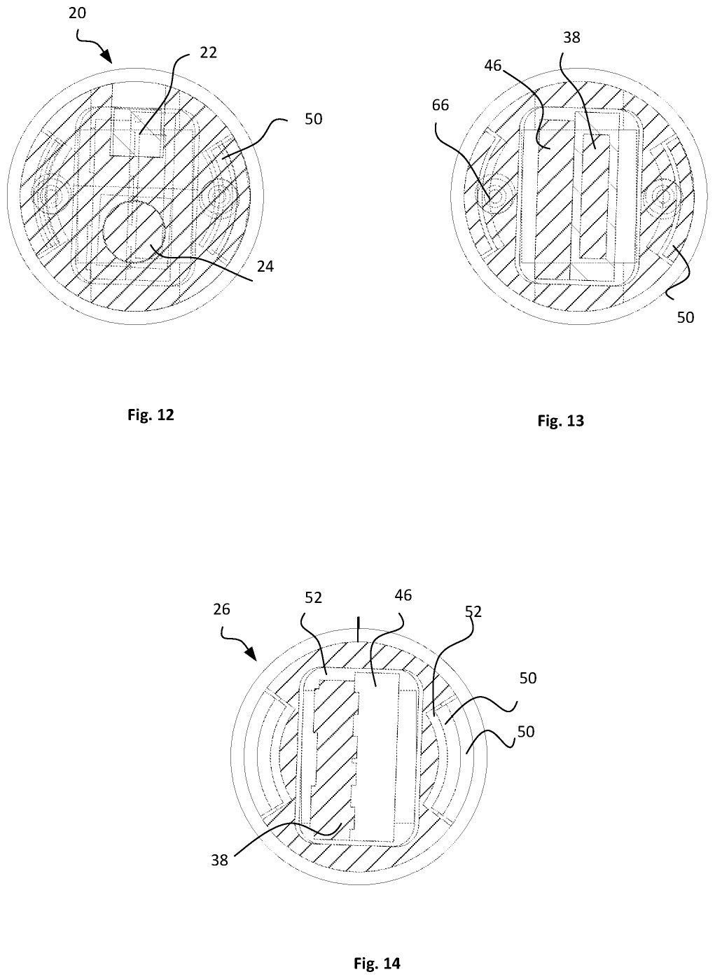

FIGS. 12-14 are circumferential cross-sectional views of the reusable optical and electrical distal end at difference respective cut-lines shown in FIG. 10 in accordance with the present disclosure.

Referring to FIGS. 12-14, in one embodiment, the end surface of distal assembly 2, distal tip 20 includes one or more light sources, an LED light 24 and camera 22. The relative axial placement of any of these components can be seen and they can be varied to suit a particular application. The light source is provided with a power source (not shown) via one of the contact points on PCB board 38.

Snap fit assembly including snap fit hook 50 and snap fit holder 52 are shown clearly in circumferential cross-sectional views. As described above, when assembled, snap fit hook 50 and snap fit holder 52 hold distal tip 20 and PCB connector 26 firmly as an integral piece during surgical procedures. The snap fit assembly can be opened during sterilization and reassembly process.

Additionally, it is contemplated that systems, devices, methods, and processes of the present application encompass variations and adaptations developed using information from the embodiments described in the following description. Adaptation or modification of the methods and processes described in this specification may be performed by those of ordinary skill in the relevant art.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

It should be understood that the order of steps or order for performing certain action is immaterial so long as the described method remains operable. Moreover, two or more steps or actions may be conducted simultaneously.

What is claimed:

1. An endoscope comprising:
an elongated bougie;
a distal tip at a distal end of the bougie, the distal tip having a sensor, a light source, and a distal tip housing forming a distal tip chamber;
an electronic connector having at least one PCB board, and at least a portion of the PCB board is nestled within the distal tip chamber, the electronic connector is disposed between the distal tip and the distal end of the elongated bougie when being assembled;
a handle abutting a proximal end of the elongated bougie.

2. The endoscope of claim 1, wherein the electronic connector includes a pair of PCB connector and power connector.

3. The endoscope of claim 1, wherein the electronic connector is a pair of USB-C connector and socket.

4. The endoscope of claim 1, wherein the handle comprises an articulation knob and the bougie has an elongated hollow core housing at least one pull wire, and when the articulation knob is maneuvered, the distal end is pulled away by the pull wire from an axial direction, causing the bougie to deflect.

5. The endoscope of claim 1, wherein the electronic connector and the distal tip have a pair of matching removably attaching means causing the distal tip and the electronic connector to be removably attached as an integral distal assembly when assembled.

6. The endoscope of claim 5, wherein the pair of matching removably attaching means is a pair of snap fit hook and snap fit holder.

7. The endoscope of claim 5, wherein the integral distal assembly is configured to be disengaged from the bougie and to be collected at a site of a medical procedure and later disassembled, sterilized, and reassembled with another new bougie.

8. The endoscope of claim 1, wherein the electronic connector has a PCB board connector housing holding at least partially the at least one PCB board, and the PCP board connector housing is configured to be removably attached to the distal tip to electronically connect the PCB board and/or power with the sensor and the light source when assembled.

9. The endoscope of claim 1, wherein the electronic connector has a PCB connector housing configured to be pulled apart from the distal tip chamber, bringing the PCB board away from the distal tip when disassembled or to be pushed into the distal tip chamber co-axially, causing the PCB board to be nestled within the distal tip chamber.

10. The endoscope of claim 1 further comprises liquid sealing meanings along abutting locations between a housing of the electronic connector and the distal tip housing.

11. The endoscope of claim 1, wherein the bougie further comprises a vertebrae column at the distal end of the bougie, and when the articulation knob is maneuvered, the distal end is pulled away from an axial direction, causing the vertebrae column to deflect.

12. The endoscope of claim 1, wherein the bougie is a passively flexible elongated tube.

13. The endoscope of claim 1, wherein the sensor is a camera.

14. The endoscope of claim 1, wherein the light source is an LED light.

15. The endoscope of claim 1, wherein the sensor and the light source are both hosted by the distal tip housing at locations the most distal from the handle.

16. A distal assembly of an endoscope comprising,
a distal tip having a sensor, a light source, and a distal tip housing forming a distal tip chamber;
an electronic connector having at least one PCB board, and at least a portion of the PCB board is configured to nestle within the distal tip chamber, the electronic connector is disposed between the distal tip and a distal end of the elongated bougie when assembled.

17. The distal assembly of the endoscope of claim 16, wherein the elongated bougie comprises a proximal end configured to be attached to an endoscope handle.

18. The distal assembly of the endoscope of claim 17, wherein the handle comprises an articulation knob and the bougies has an elongated hollow core housing at least one pull wire, and when the articulation knob is maneuvered, the distal end is pulled away from an axial direction, causing the bougie to deflect.

19. The distal assembly of the endoscope of claim 16, wherein the electronic connector and the distal tip has a pair of matching removably attaching means causing the distal tip and the electronic connector to be removably attached as an integral distal end when assembled.

20. The distal assembly of the endoscope of claim 19, wherein the pair of matching removably attaching means is a pair of snap fit hook and snap fit holder.
